(19) United States
(12) Patent Application Publication
(10) Pub. No.: US 2016/0175137 A1
(43) Pub. Date: Jun. 23, 2016

(54) A Disposable and Universal Mouth Guard

(71) Applicant: Amit Karni, Arad (IL)

(72) Inventor: Amit Karni, Arad (IL)

(21) Appl. No.: 14/896,822

(22) PCT Filed: Jun. 1, 2014
(86) PCT No.: PCT/IL2014/050488

§ 371 Date: Dec. 8, 2015

(30) Foreign Application Priority Data
Jun. 10, 2013 (IL) 226852

(51) Int. Cl. A61F 5/56

(52) U.S. Cl. CPC A61F 5/566; A61F 2005/563

(57) ABSTRACT

The invention relates to a disposable mouth guard, conformable to individual mouth structures, characterized in that it comprises compacted fibrous material comprising: end tongues suitable for improving the positioning of the device, wherein said end tongues are shaped to be wrapped around a user's gums in order to prevent the movement of the guard against the teeth.

### FIELD OF THE INVENTION

The invention relates to the field of dentistry. More particularly, the invention relates to a device suitable to be placed inside the mouth, mainly while sleeping, to prevent bruxism or any other involuntary muscular activity in the jaw area.

### BACKGROUND OF THE INVENTION

While sleeping, there can be an involuntary activity of the chewing system that includes the teeth, the muscles, the jaw, and the cartilage and the joints of the jaw. This phenomenon can also occur when a person is awake, but it is less likely. For that reason, although is it a common phenomenon, many people who suffer from bruxism during the night are sometimes unaware of its existence, until physical damage occurs.

The involuntary movement of said parts usually involves the teeth and causes teeth grinding. The friction between the upper and the lower teeth causes deformation of the outer surface of the teeth, decrease of enamel layer, and even breaking of fillings or crowns.

Another form of damage is the implications of the muscles movement. Sustained movement of the muscles can cause headaches and migraines, pain in the face, the ears, the neck, the cartilage and joints of the jaw, and can also inflict pain and bite marks on the cheeks and tongue. If the cartilage or the joints of the jaw are in fact damaged, the person who suffers from it could have functional disorders such as a difficulty in opening the mouth, unsymmetrical opening and motion of the mouth, and even unnatural noises when moving the mouth.

There are many oral devices, such as mouth guards, provided with motion-limiting means, suitable to prevent the bruxism phenomenon, but the majority of said devices need to be individually customized and manufactured. The personal adjustment process increases the price of the product and makes it less available to patients.

Another disadvantage common in prior art devices is the repeated use of the same product for a number of times, and/or for a long period of time, requiring daily cleaning for repeated use. One of the reasons for using the same device every day relates to the personal adjustment method and the individual manufacturing that increases the cost. Another reason for re-using the device is the materials of which it is made. Plastic materials for example are very common for this purpose, for their pressure endurance and because they are relatively easy to shape. The drawback of such materials, except for their cost, is the fact that they are unfriendly to the environment and not disposable. Another disadvantage is the fact that plastic materials can easily suffer from cracks as a result of friction, temperature changes and dryness. When such cracks are formed, it makes the cleaning process more difficult, and the end result less than satisfactory. Yet another disadvantage common in prior art devices is that they are best worn only after correction by dentist of balanced bite across all teeth, delaying relief of pain by weeks and even months.

It is an object of the invention to provide a device that overcomes the drawbacks of the prior art, particularly with regard to cost, suitability for every person without the need of personal adjustments, cleaning requirements, and immediate use.

Other objects and advantages of the invention will become apparent as the description proceeds.

### SUMMARY OF THE INVENTION

The invention relates to a disposable mouth guard, suitable to adjust to most individual mouth structures, characterized in that it is made of partially compacted fibrous material. By “partially compacted” it is meant to refer to material that has been compacted to an extent that it can still be further compacted by the pressure applied by human teeth. Such materials can be cotton or plastic felt, for example.

The device is adjustable to an individual's dental system shape, when light pressure by the teeth is applied thereon. It allows the device to fit relatively comfortably inside the mouth. The pressure applied on said mouth guard can be produced by closing the jaws when it is held between the upper and the lower jaws. The initial shape of the device can be flat and can also be flat on one side, and has spaces to host teeth on the other side, or can be any other shape that allows the placement of the device inside the mouth and keeps the device adjustable. The device can be moistened e.g. by water for better customization.

The length of the mouth guard is determined by the need of the patient, and can cover some or all of the teeth. There is a possibility to manufacture the device with a channel, suitable for the insertion of strengthening and/or stiffening means.

### BRIEF DESCRIPTION OF THE DRAWINGS

In the drawings:

FIG. 1A is a perspective view of the device, according to one embodiment of the invention, suitable to be placed around a front group of teeth, in the lower or upper jaw;

FIG. 1B is a top view of the device of FIG. 1A;

FIG. 2A is a perspective view of the device, according to one embodiment of the invention, suitable to be placed around all teeth, in the lower or upper jaw;

FIG. 2B is a top view of the device of FIG. 2A;

FIG. 3A is a perspective view of the device, according to one embodiment of the invention, suitable to be placed around a front group of teeth, in the lower or upper jaw. Further views of this embodiment show spaces for the teeth at the bottom of the device;

FIG. 3B is a back view of the device of FIG. 3A;

FIG. 4A is a front view of the device of FIG. 3A;

FIG. 4B is a cross-sectional view A-A of the device of FIG. 4A;

FIG. 5A is a perspective view of the device, according to one embodiment of the invention, suitable to be placed around all teeth, in the lower or upper jaw. Further views of this embodiment show spaces for the teeth at the bottom of the device;

FIG. 5B is a back view of the device of FIG. 5A;

FIG. 6A is a front view of the device of FIG. 5A;

FIG. 6B is a cross-sectional view B-B of the device of FIG. 6A; and

FIG. 6C is a cross-sectional view B-B of the device of FIG. 6A with an addition of a channel for the insertion of strengthening means.

### DETAILED DESCRIPTION OF THE INVENTION

The invention relates to dental devices that prevent damage to the teeth and chewing system, caused by involuntary muscle movement. It is highly important to produce a universal mouth guard in order to make the device available for everyone, without the hassle and high cost that are usually required when one purchases a mouth guard.

Another cost-efficient and environmentally friendly aspect of the invention is the use of disposable materials. The constant replacement of the mouth guard also contributes to mouth hygiene. It is also time-saving, and it prevents the accumulation of materials on the mouth guard and/or between the device and the teeth. The mouth guard can be used whenever desired, without the need to correct and balance the device according to the jaw bite of the patient by dentistry work.

The involuntary movement that appears in bruxism can be expressed differently with each person. Sometimes it involves only the front teeth and muscles, and when it is more severe, there is a need for a larger mouth guard that protects and prevents movement through the entire jaw. As the description proceeds, the mouth guard used to protect only the front area will be referred as “a partial guard” and the larger mouth guard will be referred as “a full guard”.

All of the embodiments presented in further descriptions are suitable to be placed around the lower teeth and gums and around the upper teeth and gums as well.

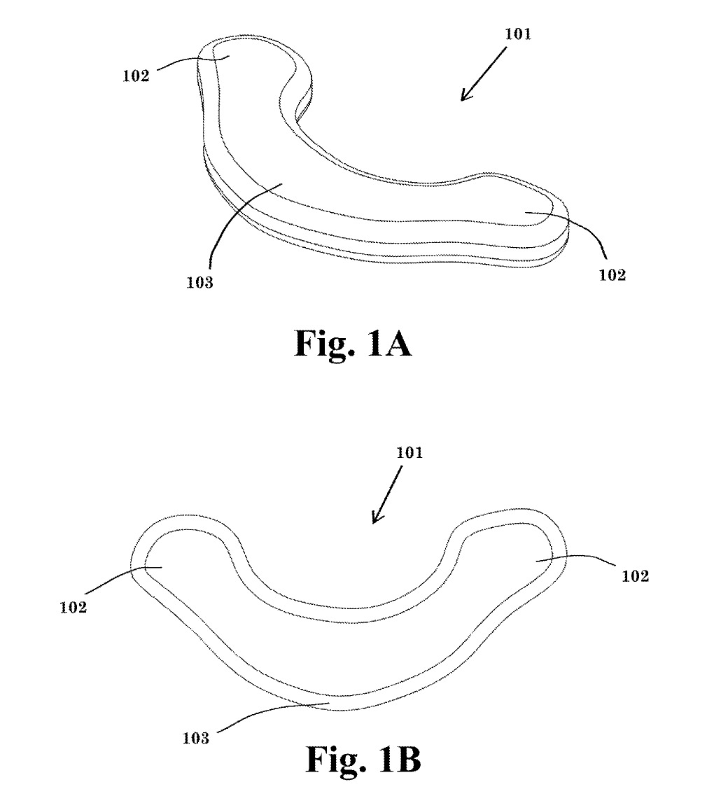

FIG. 1A shows a partial guard 101 in a perspective view, according to one embodiment of the invention. The guard 101 is flat and it is made of a material designed to take the shape of its surrounding, like fabric or felt for example, when pressure is applied on it. Such mouth guard has the ability to conform, especially when it is wet (either after being placed in the mouth or prior to the insertion into mouth, i.e., pre-wetted)—it can take the shape of its surrounding and fit in a relatively comfortable positioning inside the mouth. A mouth guard can also be partially compacted in order to take the shape of its surrounding, without reaching its maximum compression. When the patient puts the guard 101 inside his mouth with its front end 103 facing outwards, and applies pressure with his teeth, the guard is adjusted accordingly, spreading the bite force and pressure evenly across the teeth in contact with it. FIG. 1B is a top view of FIG. 1A.

The edges 102 of the guard stick out for better positioning of the guard inside the mouth. When a patient closes his mouth, those edges, hereinafter—end tongues 102 are wrapped around the gums in order to prevent the movement of the guard against the teeth.

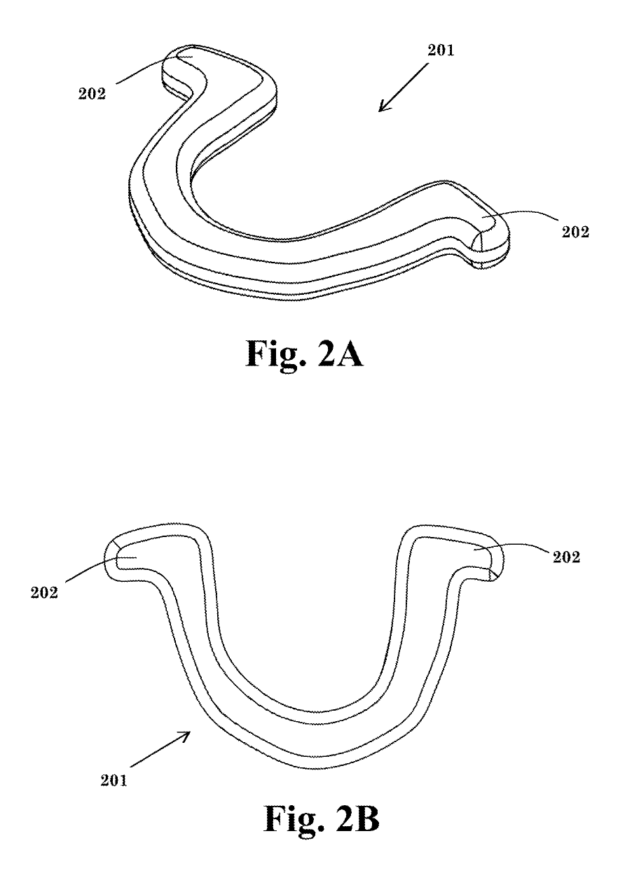

A full guard 201, according to another embodiment of the invention, is shown in a perspective view in FIG. 2A and in a top view in FIG. 2B. The guard 201 is flat and is used like the partial guard 101 inside the mouth, and it is also made of easily-shaped, compressed fibrous materials. The end tongues 202 are shaped to be wrapped around the back portion of the gums, in a way that allows the guard 201 to cover all teeth.

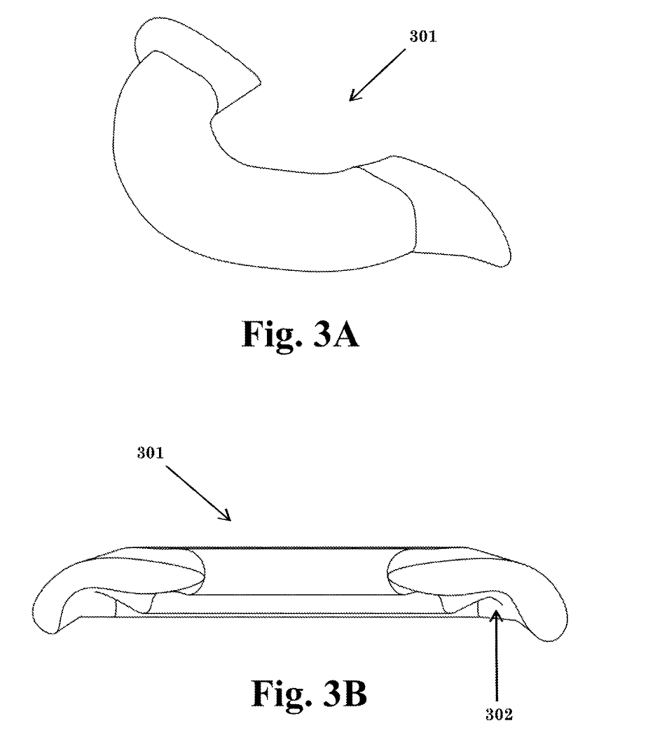

For better positioning of the device, the mouth guard can be manufactured already including one or more spaces or grooves, or a singular channel-like space, for the teeth. The space/groove allows room for the teeth, which can be convenient in some cases and provide better positioning of the guard. FIG. 3A is a schematic perspective view of such a guard 301. FIG. 3B is a back view of said guard 301, wherein spaces 302 are shown. The spaces/grooves 302 are fit to comfortably host the teeth.

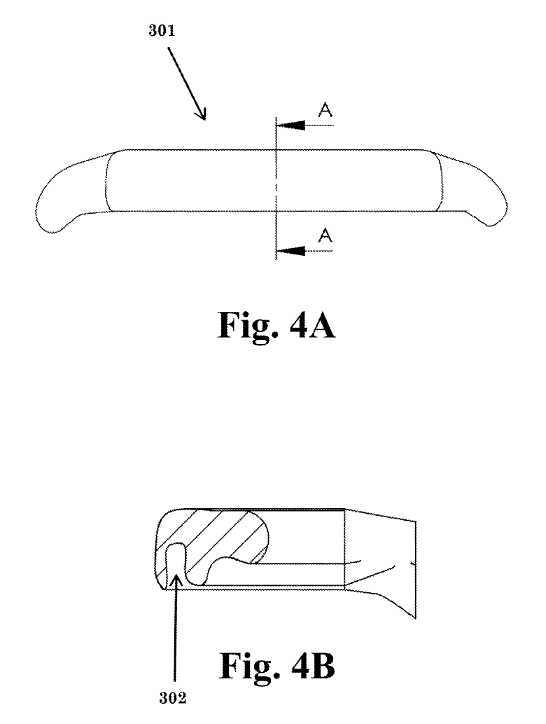

FIG. 4A is a front view of the device of FIG. 3A and FIG. 4B is a cross-sectional view A-A of FIG. 4A. The size of the spaces/grooves 302 can change along the guard 301, depending on the size of the teeth along the jaw. However, these pre-formed spaces will easily conform to the individual user's teeth shape when pressure is applied thereon by the teeth.

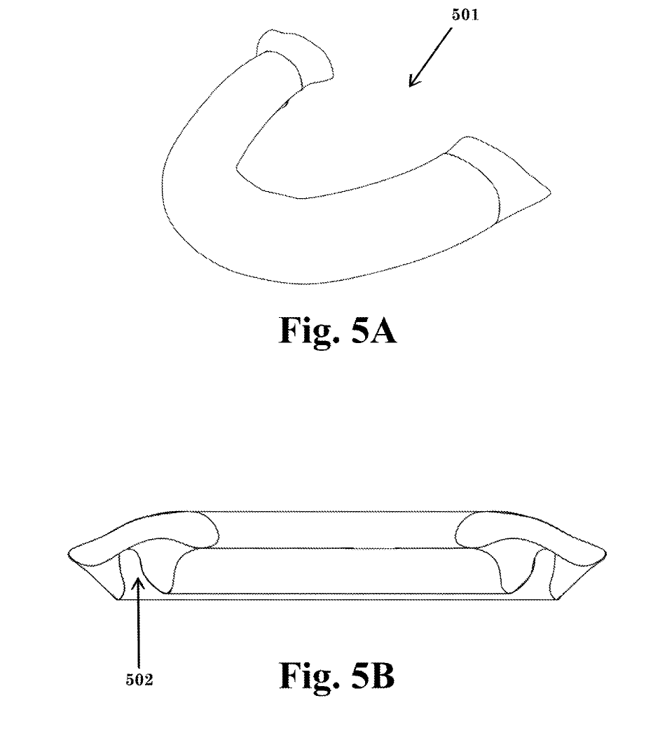

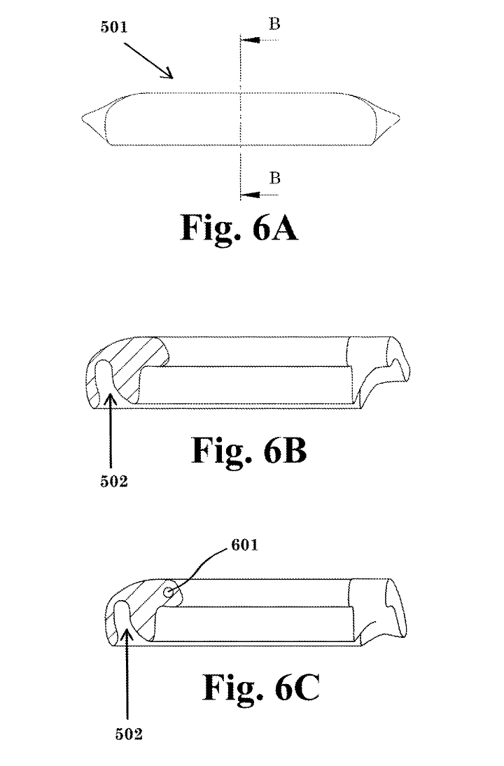

FIGS. 5A-B and 6A-C show another embodiment of the invention. The guard 501 covers all the teeth, like the full guard 201, but also has a space/groove for the teeth 502, like the guard 301 of FIG. 3B. FIG. 5B is a back view and FIG. 6A is a front view of the device of FIG. 5A.

FIGS. 6C and 6C are both cross-sectional views B-B of the device of FIG. 6A, but in FIG. 6C there is a channel 601 for the insertion of strengthening or stiffening means, such as a plastic wire or other structural strengthening element. The device can be manufactured with or without the channel 601 and the strengthening means, but it is recommended when the bruxism is severe, because the movements are extreme and there is a need for additional restraint.

All the above description has been provided for the purpose of illustration and is not meant to limit the invention in any way. The invention presents significant advantages over the existing art, for example, the universal use, and the use of disposable materials.

1. A disposable mouth guard, conformable to individual mouth structures, characterized in that it is made of compacted fibrous material that has been compacted to an extent that it can still be further compacted by the pressure applied by human teeth, comprising: end tongues suitable for improving the positioning of the device, wherein said end tongues shaped to be wrapped around a user's gums in order to prevent the movement of the guard against the teeth.

2. A mouth guard according to claim 1, wherein the fibrous material is cotton.

3. A mouth guard according to claim 1, wherein the fibrous material is a fibrous plastic material.

4. A mouth guard according to claim 1, wherein the fibrous material is partially compacted.

5. A mouth guard according to claim 1, wherein the material from which the device is produced, is processed into felt.

6. A mouth guard according to claim 1, that is adjustable to an individual's dental system shape, when pressure is applied thereon.

7. A mouth guard according to claim 6, wherein the pressure applied on said mouth guard is produced by closing the jaws when it is held between the upper and the lower jaws.

8. A mouth guard according to claim 1, wherein its initial shape is flat.

9. A mouth guard according to claim 1, wherein its initial shape is flat on one side, and has spaces to host teeth on the other side.

10. A mouth guard according to claim 1, wherein the length of said mouth guard is suitable to cover some of the teeth.

11. A mouth guard according to claim 1, wherein the length of said mouth guard is suitable to cover all teeth.

12. A mouth guard according to claim 1, provided with a channel, suitable for the insertion of strengthening means.

13. A mouth guard according to claim 1, which is suitable to be pre-wetted for ease of customization.
